(19) United States
(12) Patent Application Publication
(10) Pub. No.: US 2004/0225309 A1
(43) Pub. Date: Nov. 11, 2004

(54) Apparatus and method for dermal tissue harvesting

(76) Inventors: Elof Eriksson, Wellesley, MA (US); Christian Baker, Weymouth, MA (US); W. Robert Allison, Sudbury, MA (US)

Correspondence Address: QUARLES & BRADY LLP, 411 E. WISCONSIN AVENUE, SUITE 2040, MILWAUKEE, WI, 53202-4497, US

(21) Appl. No.: 10/775,449

(22) Filed: Feb. 10, 2004

Related U.S. Application Data
(60) Provisional application No. 60/446,208, filed on Feb. 10, 2003.

(51) Int. Cl. A61B 17/32; B26B 21/08

(52) U.S. Cl. 606/167; 30/51

(57) ABSTRACT

A dermal tissue harvesting apparatus is provided having a handle attached to a housing. The housing defines a base having a forward base portion spaced apart from a rearward base portion by a slot extending through the base. The forward base portion is offset higher than the rearward base portion. A ramp is provided that supports a cutting blade at an angle relative to the base. The blade defines a cutting edge that extends through the slot. The blade depth can either be fixed on the ramp, or adjustable.

### CROSS-REFERENCE TO RELATED APPLICATIONS

This claims priority to U.S. provisional patent application serial No. 60/446,208, filed Feb. 20, 2003, the disclosure of which is hereby incorporated by reference.

### STATEMENT REGARDING FEDERALLY SPONSORED RESEARCH OR DEVELOPMENT

### BACKGROUND OF THE INVENTION

The invention relates to an apparatus and method for harvesting dermal tissue from a dermal tissue donor site. More particularly, the invention relates to an apparatus and method for obtaining the dermal tissue.

Skin grafting traditionally involves removing a thin slice of dermal tissue from a donor site on a human or non-human animal. The thin tissue slice is transplanted to a recipient site, typically a wound site, that is covered or partially covered by the grafted tissue. In some cases, the tissue is processed, for example by meshing, before being applied to the recipient site. Typically, the donor site and recipient site are on the same individual animal.

Various devices are used to obtain dermal tissue from the donor site. Dermatomes generally employ a thin blade to cut slices of skin from the patient. In some devices, an oscillating motor moves the blade rapidly from side to side. These devices can require a high degree of skill and experience from the operator to insure the quality of the harvest and the safety of the patient. The cost of these devices, including supplies, maintenance, and training, can be expensive. It is therefore an object of the present invention to provide a simpler harvesting device that is inexpensive, disposable, and easy to use. Additional objects of the present invention include a high quality of harvest and greater safety for the patient.

### SUMMARY OF THE INVENTION

In one aspect, the present invention provides an apparatus for harvesting dermal tissue. The apparatus includes a housing having a base that defines spaced apart substantially planar forward and rearward portions defining a slot therebetween. The forward base portion is offset higher than the rearward base portion. A support ramp is attached to the housing and defines an angle with respect to the base. A cutting blade is coupled to the support ramp and defines a cutting edge extending through the slot to a depth lower than the forward base portion.

The foregoing and other aspects of the invention will appear from the following description. In the description, reference is made to the accompanying drawings which form a part hereof, and in which there is shown by way of illustration, and not limitation, a preferred embodiment of the invention. Such embodiment does not necessarily represent the full scope of the invention, however, and reference must therefore be made to the claims herein for interpreting the scope of the invention.

### BRIEF DESCRIPTION OF THE DRAWINGS

FIG. 1 is a perspective view of a dermal tissue harvesting apparatus carrying a handle and a cutting blade housing in accordance with the preferred embodiment;

FIG. 2 is a perspective view of the cutting blade housing illustrated in FIG. 1 carrying a cutting blade assembly;

FIG. 3 is an exploded assembly view of the components of the cutting blade assembly illustrated in FIG. 3;

FIG. 4 is a sectional side elevation view of the cutting blade assembly illustrated in FIG. 2;

FIG. 5 is a sectional side elevation view of a cutting blade assembly constructed in accordance with an alternate embodiment of the invention;

FIG. 6 is a sectional end elevation view of the cutting blade assembly taken along line 6-6 of FIG. 5;

FIG. 7 is a perspective view of an adjustable cutting assembly constructed in accordance with an alternate embodiment of the invention;

FIG. 8 is an exploded assembly view of the cutting assembly illustrated in FIG. 7;

FIG. 9 is a side elevation view of the cutting assembly taken along line 9-9 of FIG. 7;

FIG. 10 is a sectional side elevation view of the harvesting apparatus illustrated in FIG. 5 during operation; and

FIG. 11 is a perspective view of the harvesting apparatus illustrated in FIG. 1 constructed in accordance with an alternate embodiment of the invention.

### DESCRIPTION OF THE PREFERRED EMBODIMENTS

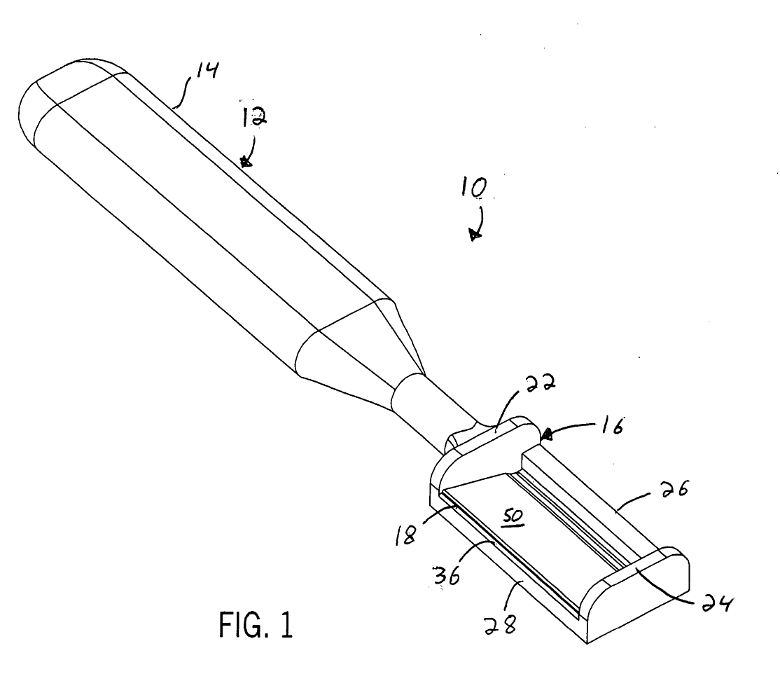

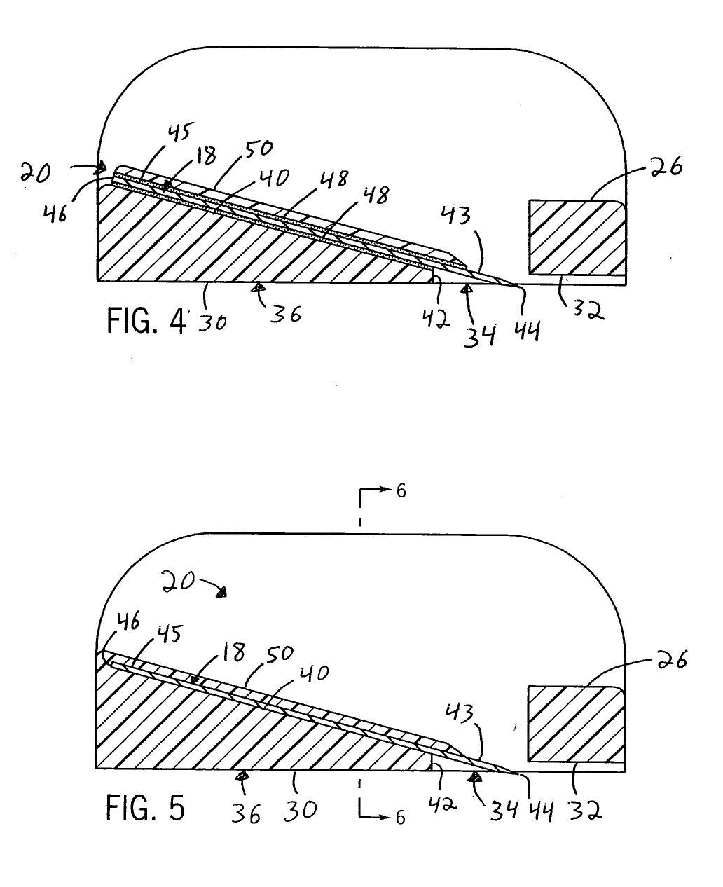

Referring to FIGS. 1-4, a disposable dermal tissue harvesting apparatus 10 includes a housing 12 defining a handle 14 at one end that is connected to a cutting blade housing 16 at its opposite end. Preferably handle 14 and housing 16 comprise an integral molded member formed from a suitable plastic. Handle 14 is preferably sized and shaped to be comfortably gripped by a user when harvesting tissue of a patient.

Housing 16 includes a pair of opposing longitudinally extending side walls 22 and 24. Sidewalls 22 and 24 are connected at their outer ends to opposing laterally extending front and rear end walls 26 and 28, respectively. It should be appreciated that the term “front” and “rear,” and their synonymous designations, are used throughout the description with reference to the direction of movement in the longitudinal direction when harvesting tissue. Rear end wall 28 defines a flat rear base portion 30 that extends horizontally forwardly from the bottom edge of wall 28.

As illustrated in FIG. 1, handle 14 extends outwardly from side wall 22. Accordingly, as will become further apparent from the description below, apparatus 10 is configured to be engaged by the right hand of a user. The present invention recognizes, however, that handle 14 could alternatively extend outwardly from wall 24, and thus more easily engaged by the left hand of a user. Alternatively still, referring to FIG. 11, harvesting apparatus 10 can enable handle 14 to be attached to either wall 22 or 24 by the user. In particular, a mounting bracket 27 can connect to both walls 22 and 24 (illustrated in FIG. 11 as extending from wall 24) that is sized to engage a corresponding groove formed in handle 14. A stop or a catch 29 can be provided proximal the lower end of bracket 27 that engages a corresponding lip on handle 14 to lock the handle in place. It should be appreciated, however, that any alternative mechanical fastener suitable for connecting (and preferably removably connecting) handle 14 to walls 22 and 24 is contemplated by the present invention.

Referring again to FIGS. 1-4, rear end wall 28 defines the rear end of a cutting blade support ramp 36. An upper ramp surface 40 depends downwardly and forwardly from rear end wall 28, and terminates at a front end wall 42 that extends vertically between the ramp surface 40 and rear base portion 30. Ramp surface 40 defines an angle with respect to base portion 30 that is preferably within the range of 1 to 45 degrees, more preferably 15-40 degrees, and more preferably still 15-30 degrees.

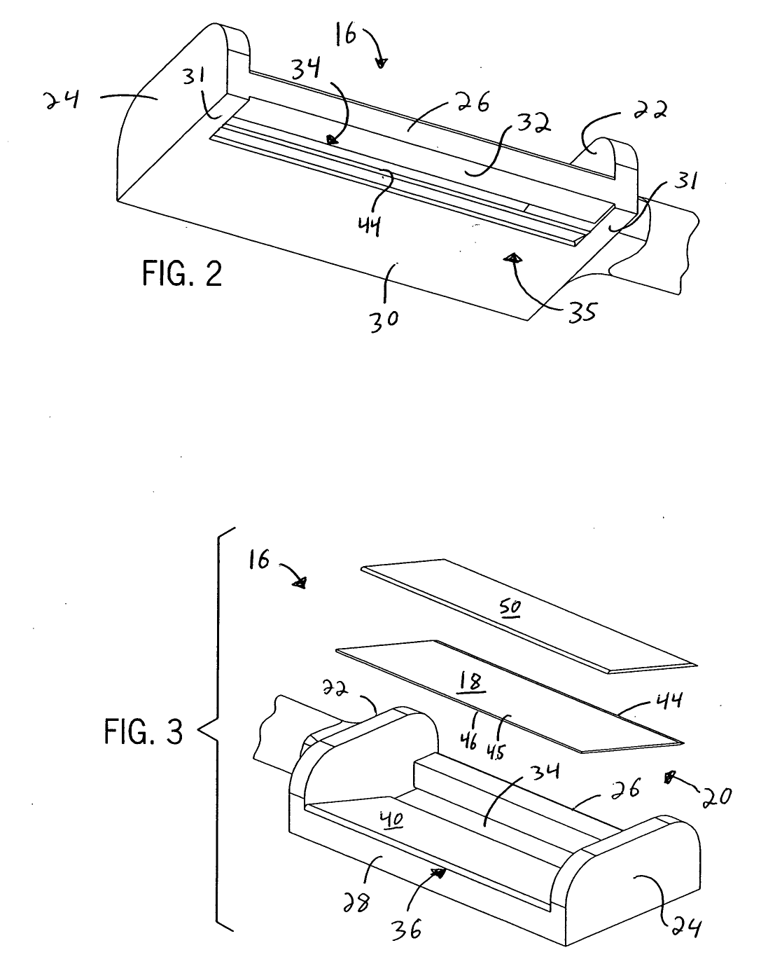

A flat forward base portion 32 extends rearwardly from the lower end of front end wall 26, and is slightly raised with respect to rear base portion 30. Base portion 32 extends horizontally rearwardly from front end wall 26 towards rear end wall 28. Base portion 32 terminates short of base portion 30, such that a slot 34 is defined between the rear terminal end of base portion 32 and ramp front end wall 42. Slot 34 extends laterally between sidewalls 22 and 24. It should be appreciated that base portions 30 and 32 together define a base 35 of housing 16 (See FIG. 2). As further illustrated in FIG. 2, rear base portion 30 defines forward extension members 31 that extend forwardly to front wall 26 at the opposing laterally outer ends of base portion 30 and are vertically aligned with end walls 22 and 24. The lateral distance between extension members 31 defines the length of slot 34.

Housing 16 carries a cutting assembly 20, which includes a cutting blade 18 that defines a forward end 43 presenting a cutting edge 44, and a rear end 45 presenting a non-cutting edge 46. Blade 18 is disposed on ramp surface 40 at an angle relative to the base 35 which is determined by the ramp angle. Cutting edge 44 protrudes through the slot 34 and crosses the plane of front base portion 32, and also preferably (though not necessarily) crosses the plane of rear base portion 30. It will thus be appreciated that the ramp angle determines an angle of attack of the cutting blade 18, which is selected based on the skin type to be harvested. Non-cutting edge 46 can face away from the slot 34.

In accordance with the preferred embodiment, cutting blade 18 is fastened to ramp surface 40 via an adhesive 48, bolt, rivet, or alternative fastener at a position that locates cutting edge 44 at a fixed predetermined depth relative to base 35, it being appreciated that the depth determines the thickness of tissue to be harvested. The present invention envisions that a plurality of disposable tissue harvesting apparatuses 10 of the type described above can be provided having varying fixed predetermined blade depths suitable for specific tissue harvesting operations. The cutting edge 44 of the blade can further be sized and shaped to accommodate a particular use, according to, e.g. the contour of the dermal tissue to be harvested, the desired length of the a graft to be harvested or the size of the recipient site. For instance, cutting edge 44 can be straight or can be provided with, e.g. a scalloped edge.

A retaining plate 50 can be attached to the upper surface of blade 18. Plate 50 preferably extends between side walls 22 and 24 (or is otherwise sized to cover blade 18), and extends forwardly so as to cover the majority of blade 18 while leaving forward end 43 exposed. Retaining plate 50 can be made out of any material, for example plastic, and is provided for aesthetic purposes, and also to protect the blade 18 from being damaged during storage. Retaining plate 50 is preferably attached directly to blade 18 using adhesive 48, bolt, rivet, or alternative fastener.

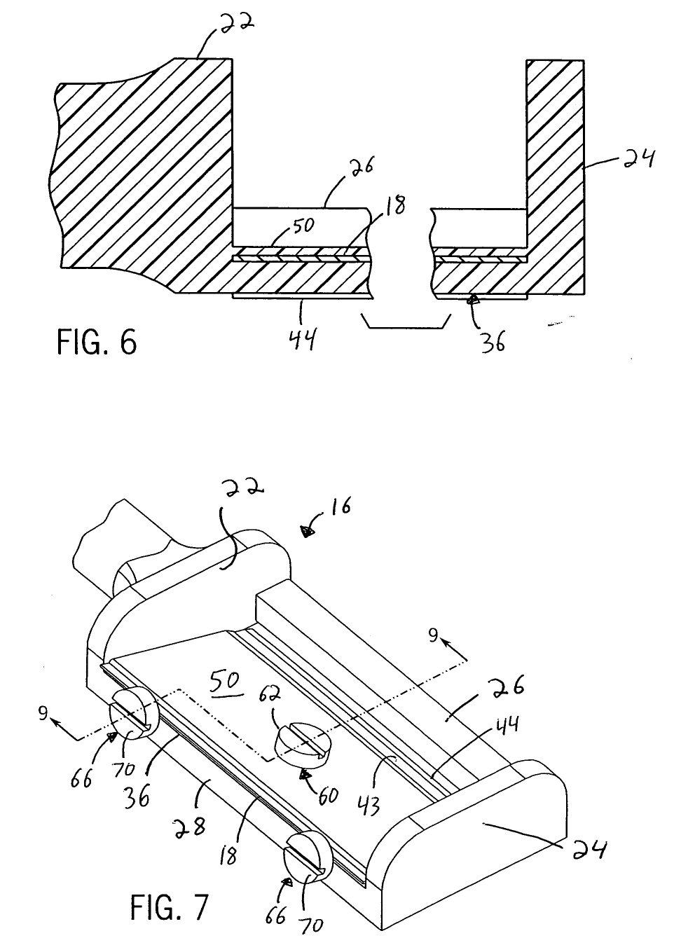

Alternatively, referring to FIGS. 5 and 6, housing 16 can be molded around any suitable edge or edges of blade 18 in accordance with any known method without requiring adhesive to bond the blade to the housing 16. For instance, the side and back ends of the blade 18 can be encapsulated by the molded housing 16. In accordance with this embodiment, blade 18 becomes attached to the housing 16 during fabrication of the housing. Furthermore, the molded housing could extend partially or entirely across upper blade surface 45. Accordingly, retaining plate 50 can be defined by the housing material molded around blade 18 during fabrication of housing 16, and thus does not require adhesive 48.

The tissue harvesting apparatus 10 described above is relatively inexpensive to produce, and is thus suitable to be discarded after a harvesting operation is performed. However, the present invention recognizes that it may be desirable for harvesting apparatus 10 to have a blade 18 of adjustable depth. Furthermore, it may be desirable for harvesting apparatus 10 to be reusable.

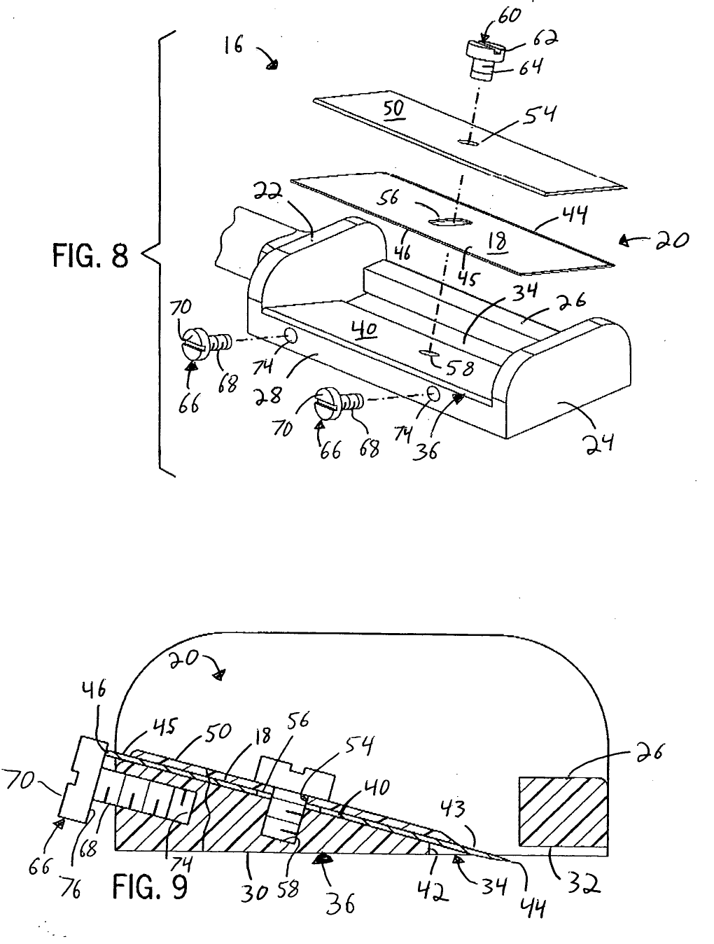

In particular, referring to FIGS. 7-9, an aperture 54 extends through retaining plate 50 that is laterally aligned with a corresponding slot 56 extending forwardly through blade 18. A corresponding threaded aperture 58 extends downwardly into ramp 36 at an angle preferably normal to ramp surface 40. A retaining screw 60 is provided having a screw head 62 and a threaded shaft 64. Aperture 54 is preferably sized substantially equal to shaft 64 such that shaft 64 can extend through plate 50 without allowing plate 50 to translate relative to screw 60. Side walls 22 and 24 also prevent translation of plate 50 relative to screw 60 and housing 16, and further prevent rotation of plate 50 relative to housing 60. Slot 56 defines a lateral thickness that is substantially equal to (or slightly greater than) the diameter of shaft 64 to accommodate screw 60 while preventing lateral blade translation relative to screw 60. Preferably side walls 22 and 24 also prevent lateral translation of blade 18 relative to screw 60 and housing 16, and further prevent rotation of blade 18 about screw 60 relative to housing 16. Slot 56 extends longitudinally a distance substantially greater than that diameter of shaft 64 to enable forward and backwards translation of blade 18 relative to housing 16.

One or more (a pair, as illustrated) depth setting blade stops 66 is provided, and illustrated as a screw having a threaded shaft 68 connected to a screw head 70. A corresponding threaded aperture 74 extends through the rear surface 28 of end wall 28 and is sized to receive threaded shaft 68. Each aperture 74 preferably extends parallel to ramp surface 40. Alternatively still, apertures 74 can extend normal with respect to rear surface 28, or in any other direction that enables screw heads 70 to engage the non-cutting edge 46 of blade 18. When screws 66 are rotatably inserted into apertures 74, the inner surfaces 76 of screw heads 70 define a depth relative to end wall 28 that, in turn, defines the depth of cutting edge 44.

During operation, retaining screw 60 is loosened until the blade 18 is held loosely against the support ramp 36 by the retaining plate 50 to enable relative movement between the blade 18 and housing 16. Next, depth setting screws 66 are rotated clockwise (i.e., tightened) to urge blade 18 down the support ramp 36 towards slot 34, thereby increasing the protrusion of cutting edge 44 through the slot 34. When screws are rotated counterclockwise (i.e., loosened), blade 18 can be urged up the support ramp 36 and away from the slot 34 until the non-cutting edge 46 contacts the inner surfaces 76 of screw heads 70, leaving less of the blade 18 protruding through the slot 34. Side walls 22 and 24 provide guides that maintain the blade 18 and cutting edge 44 in the proper orientation while adjusting the blade depth. Once the desired cutting depth is set, retaining screw 60 is tightened to bias retaining plate 50 against the blade 18, thereby retaining blade 18 in a fixed position against ramp surface 40.

Alternatively, a washer (not shown) could replace retaining plate 50 to provide a variable force against blade 18 as screw 60 is loosened and tightened. Alternatively still, a washer (not shown) could be provided in combination with screw 66 in the usual manner having a sufficiently large diameter to engage the non-cutting edge 46 of blade 18. Alternatively still, any member having a position that is adjustable relative to housing 12 and/or 16 that can provide an adjustable stop to set the blade depth is contemplated by the present invention.

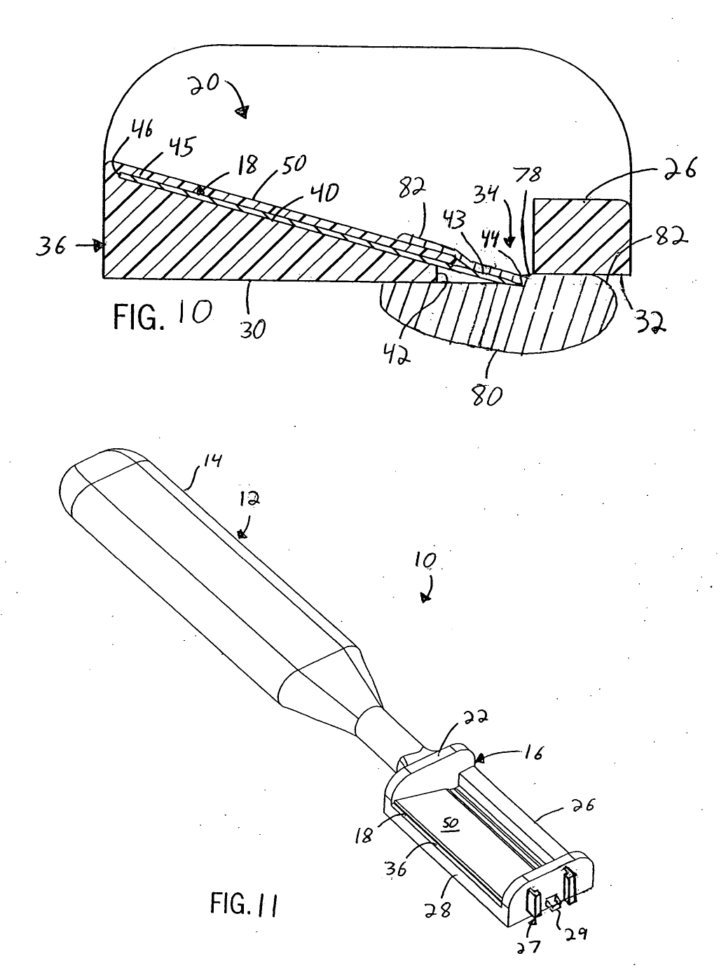

Referring now to FIG. 10, once the depth is set (if harvester 10 provides an adjustable blade depth), and the retaining screw 60 tightened, the user grasps the handle 14 (which extends generally in a direction parallel to cutting edge 44) and brings the base 35 into contact with the donor dermal harvest site 78, establishing the proper orientation and position of the cutting blade 18 relative to the dermal tissue 80. When the apparatus 10 is properly positioned against the dermal tissue 80, the cutting edge 44 penetrates into the tissue to a consistent depth and yields harvested dermal tissue 82 having a generally uniform thickness, which can be selected either by employing an adjustable blade, or by selecting a fixed blade that yields a cut at a desired depth, as described above. A preferable depth of penetration can range from approximately 0.005 in to 0.030 in, and more preferably between 0.010 in to 0.020 in.

The user oscillates the device forward and backward along a lateral path. This motion causes the cutting blade to move in a corresponding manner. The length of travel of each oscillation can be determined by the user according to the user's requirements for the size of the skin harvest. At the same time the user moves the device in a longitudinal direction 90 degrees to the direction of the oscillation, thus causing the oscillating cutting blade to move into and along the skin at the harvest site. As the dermal tissue is harvested, the tissue enters into the slot 34, passes along and up the support ramp 36 and is received in the housing 16. Ramp 36 thus functions as a collection area for the harvested piece of skin.

Optionally, the ramp can be provided with side guides for directing the harvested dermal tissue up the ramp 36 and for holding the tissue away from the ramp surface 40, thereby facilitating retrieval of the tissue with forceps. Other appropriate ramp modifications can be envisioned by the skilled artisan.

When a sufficient amount of tissue has been harvested, the user can rotate the handle 14 to induce pressure on the back end of the housing 12. This urges cutting edge 44 towards the dermal surface 82, eventually cutting the harvested dermal tissue from the donor site, and leaving the harvested tissue in the collection area at the ramp. It should be appreciated that FIG. 10 illustrates the harvesting apparatus 10 in accordance with only one embodiment of the invention, however any of the embodiments illustrated and described herein can operate substantially as described above.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements included within the spirit and scope of the invention, as set forth by the appended claims.

1. An apparatus for harvesting dermal tissue, comprising:
a housing having a base and at least one side, the base having spaced apart substantially planar forward and rearward portions defining a slot therebetween, wherein the forward base portion is offset higher than the rearward base portion;
a support ramp attached to the housing defining an angle with respect to the base; and
a cutting blade coupled to the support ramp, the cutting blade defining a cutting edge extending through the slot to a depth lower than the forward base portion.

2. The apparatus as claimed in claim 1, wherein the blade presents an upper surface, further comprising a retaining plate coupled to the upper blade surface.

3. The apparatus as recited in claim 1, wherein the blade is in a fixed position relative to the housing.

4. The apparatus as recited in claim 3, wherein the blade is attached to the housing.

5. The apparatus as recited in claim 3, wherein the housing is molded around the blade.

6. The apparatus as recited in claim 3, further comprising a depth adjustment mechanism operable to adjust the depth of the cutting edge.

7. The apparatus as recited in claim 6, wherein the adjustment mechanism comprises a blade stop coupled to the housing engaging the blade at a location opposite the cutting edge.

8. The apparatus as recited in claim 7, wherein the stop comprises a screw that is threadedly attached to the housing.

9. The apparatus as recited in claim 7, wherein the adjustment mechanism further comprises a retaining member operable to increase and decrease a force biasing the blade against the ramp.

10. The apparatus as recited in claim 1, further comprising a handle attached to the housing.

11. The apparatus as recited in claim 1, wherein the handle extends in a direction generally parallel to the cutting edge of the blade.

12. The apparatus as recited in claim 1, wherein the ramp defines an angle between 1 and 45 degrees relative to the base.

13. A method for harvesting dermal tissue from a donor dermal surface site, the method comprising the steps of:
contacting the donor site with an apparatus of claim 1 such that the base of the apparatus is generally parallel to the dermal surface and the cutting blade penetrates into the tissue;
oscillating the device forward and backward along a path at the site while moving the apparatus in a direction 90 degrees relative to the oscillation path so that the cutting blade moves into and through the skin at the harvest site until harvested dermal tissue enters into the slot; and
cutting the harvested dermal tissue from the donor site.
